(19) United States
(12) Patent Application Publication
(10) Pub. No.: US 2009/0012085 A1
(43) Pub. Date: Jan. 8, 2009

(54) DOSAGE FORMS AND METHODS OF TREATMENT USING A TYROSINE KINASE INHIBITOR

(76) Inventors: Charles Michael Baum, San Diego, CA (US); Nicoletta Maria Brega, Pavia (IT); Alfonso Gentile, Kent (GB); Yazdi Kersi Pithavala, San Diego, CA (US)

Correspondence Address: PFIZER INC, 10555 SCIENCE CENTER DRIVE, SAN DIEGO, CA, 92121, US

(21) Appl. No.: 12/067,150

(22) PCT Filed: Sep. 12, 2006
(86) PCT No.: PCT/IB06/02754

§ 371 Date: Sep. 12, 2008

Related U.S. Application Data
(60) Provisional application No. 60/719,119, filed on Sep. 20, 2005.

(51) Int. Cl. A61K 31/5377; A61P 35/00; A61P 27/02

(52) U.S. Cl. 514/235.2

(57) ABSTRACT

This invention provides dosage forms of a compound of formula 1, 5-[(Z)-(5-fluoro-2-oxo-1,2-dihydro-3H-indol-3-ylidene)methyl]-N-[(2S)-2-hydroxy-3-morpholin-4-ylpropyl]-2,4-dimethyl-1H-pyrrole-3-carboxamide, or pharmaceutically acceptable salts or solvates thereof. The invention further provides methods of treating abnormal cell growth in a patient, such as cancers, by administering the dosage forms to the patient. The invention further provides methods of treating an angiogenesis- or VEGF-related ophthalmic disorder in a patient, by administering the dosage form to the patient.

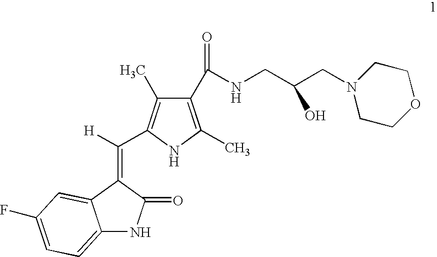

This application claims the benefit of U.S. Provisional Application No. 60/719,119, filed Sep. 20, 2005, the disclosure of which is incorporated herein by reference in its entirety.

### FIELD OF THE INVENTION

This invention provides dosage forms of a compound of formula 1, 5-[(Z)-(5-fluoro-2-oxo-1,2-dihydro-3H-indol-3-ylidene)methyl]-N-[(2S)-2-hydroxy-3-morpholin-4-ylpropyl]-2,4-dimethyl-1H-pyrrole-3-carboxamide, or pharmaceutically acceptable salts or solvates thereof. The invention further provides methods of treating abnormal cell growth in a patient, such as cancers, by administering the dosage forms to the patient. The invention further provides methods of treating an angiogenesis- or VEGF-related ophthalmic disorder in a patient, by administering the dosage form to the patient.

### BACKGROUND

The compound 5-[(Z)-(5-fluoro-2-oxo-1,2-dihydro-3H-indol-3-ylidene)methyl]-N-[(2S)-2-hydroxy-3-morpholin-4-ylpropyl]-2,4-dimethyl-1H-pyrrole-3-carboxamide, represented by formula 1,

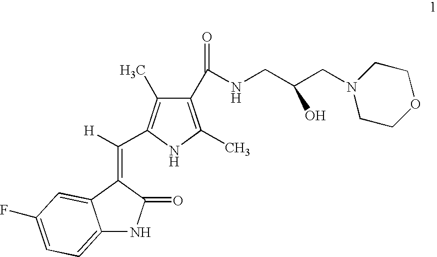

is a potent, selective oral inhibitor of receptor tyrosine kinases (RTKs) involved in signaling cascades that trigger tumor growth, progression and survival. In vivo studies have shown that this compound has anti-tumor activity in diverse preclinical solid and hematopoietic cancer xenograft models. This compound, its preparation and use are further described in U.S. Pat. No. 6,653,308, WO03/070723 (US 2003/0092917) and WO2005-033098 (US 2005-0118255). Preferred formulations of compound 1 are disclosed in WO 04/024127 (US 2004/229930). The combination therapy of compound 1 is disclosed in WO 04/045523 (US 2004/152,759). Dosage forms and methods of treatment of another selective inhibitor of RTKs are disclosed in U.S. Patent Publication No. 2005/0182122. The disclosures of these references are incorporated herein by references in their entireties.

### SUMMARY OF THE INVENTION

The invention provides dosage forms and methods of treatment using a compound of formula 1, or a pharmaceutically acceptable salt or solvate thereof:

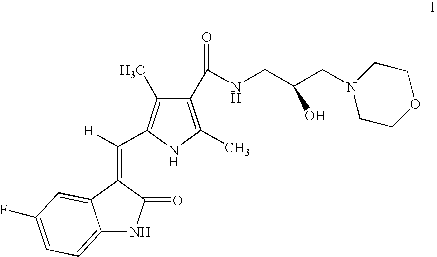

which can be systematically named as 5-[(Z)-(5-fluoro-2-oxo-1,2-dihydro-3H-indol-3-ylidene)methyl]-N-[(2S)-2-hydroxy-3-morpholin-4-ylpropyl]-2,4-dimethyl-1H-pyrrole-3-carboxamide.

In one embodiment, the present invention relates to a method of treating abnormal cell growth in a patient, comprising administering to the patient a compound of formula 1:

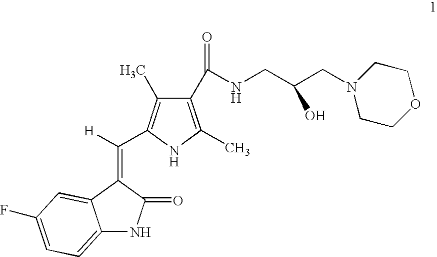

or a pharmaceutically acceptable salt or solvate thereof, or a mixture thereof, in an amount of from 5 to 300 mg free base equivalent per day. In particular, the abnormal cell growth is cancer. Even more particularly, the cancer is selected from the group consisting of a gastrointestinal stromal tumor, renal cell carcinoma, biliary cell carcinoma, thyroid carcinoma, colon adenocarcinoma, alveolar soft tissue carcinoma, thymoma, breast cancer, colorectal cancer, non-small cell lung cancer, a neuroendocrine tumor, small cell lung cancer, mastocytosis, glioma, sarcoma, acute myeloid leukemia, prostate cancer, lymphoma, and pancreatic cancer. Further more particularly, the cancer is selected from the group consisting of renal cell carcinoma, biliary cell carcinoma, thyroid carcinoma, colon adenocarcinoma, alveolar soft tissue carcinoma and thymoma.

In a further embodiment, for any of the methods or dosage forms as described herein, the pharmaceutically acceptable salt is a maleate salt.

In a further embodiment of the methods described herein, the amount of a compound of formula 1 is from 50 to 250 mg free base equivalent. For example, the amount can be 50, 75, 100, 125, 150, 175, 200, 225 or 250 mg free base equivalent. More particularly, the amount is from 100 to 200 mg free base equivalent. For example, the amount can be 100, 110, 120, 130, 140, 150, 160, 170, 180, 190 or 200 mg free base equivalent. Still more particularly, the amount is 150 mg free base equivalent. Still more particularly, the amount is 200 mg free base equivalent.

In a particular aspect, any of the amounts described herein of the compound of formula 1 is administered on a continuous dosing schedule. More particularly, the amount is administered once per day on a continuous dosing schedule. Also more particularly, the amount is administered twice per day on a continuous dosing schedule. In a further aspect, the amount is administered on an intermittent dosing schedule. In particular, the amount is administered once per day during the treatment period. Also in particular, the amount is administered twice per day during the treatment period. More particularly, the intermittent dosing schedule comprises a treatment period of from 2 to 4 weeks and a rest period of from 1 to 2 weeks. Even more particularly the intermittent dosing schedule is a 4/1 dosing schedule. Still further, the intermittent dosing schedule is a 4/2 dosing schedule. Still further, the intermittent dosing schedule is a 3/1 dosing schedule.

The present invention also provides a method of treating an angiogenesis- or VEGF-related ophthalmic disorder in a patient, comprising administering to the patient a compound of formula 1, or a pharmaceutically acceptable salt or solvate thereof, or a mixture thereof, in an amount of from 5 to 300 mg free base equivalent per day. In one aspect, the ophthalmic disorder is age related macular degeneration, choroidal neovascularization, retinopathy, retinitis, uveitis, retinal vein occlusion, iris neovascularization, corneal neovascularization, macular edema, or neovascular glaucoma.

The present invention further relates to a dosage form comprising a compound of formula 1:

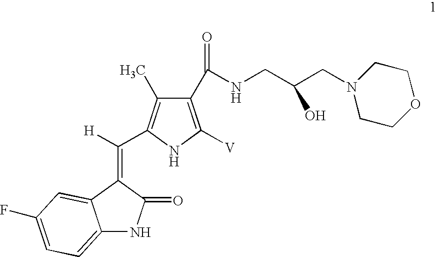

or a pharmaceutically acceptable salt or solvate thereof, or a mixture thereof, in an amount of from 5 to 300 mg free base equivalent. In one particular embodiment, the amount is from 25 to 300 mg free base equivalent. More particularly, the amount is from 50 to 250 mg free base equivalent. For example, the amount can be 50, 75, 100, 125, 150, 175, 200, 225 or 250 mg free base equivalent. Still more particularly, the amount is from 100 to 200 mg free base equivalent. For example, the amount can be 100, 110, 120, 130, 140, 150, 160, 170, 180, 190 or 200 mg free base equivalent. Even further the amount is 150 mg free base equivalent. Even further, the amount is 200 mg free base equivalent. The dosage form is suitable for administration to a mammal, such as a human, particularly for use in the treatment of any of the disorders described herein, such as abnormal cell growth, including cancers, particularly the cancers described herein, and angiogenesis- or VEGF-related ophthalmic disorders.

For any of the dosage forms described herein, in one aspect the dosage form is an oral dosage form. In a further aspect the dosage form is an intravenous dosage form. In a further aspect, for any of the dosage forms as described herein, the pharmaceutically acceptable salt is a maleate salt.

In a further aspect of the present invention is a dosage form, comprising a compound of formula 1:

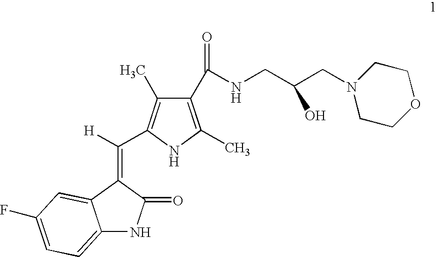

or a pharmaceutically acceptable salt or solvate thereof, or a mixture thereof, in an amount effective to provide a maximum total plasma concentration in said mammal of no more than 1,000 ng/mL of free base equivalent of the compound of formula 1. In one embodiment the maximum total plasma concentration is from 50 to 1,000 ng/mL. Even further, the maximum total plasma concentration is from 75 to 900 ng/mL. Even further, the maximum total plasma concentration is from 100 to 900 ng/mL. Even further, the maximum total plasma concentration is from 150 to 900 ng/mL. Even further, the maximum total plasma concentration is from 175 to 875 ng/mL. Even further, the maximum total plasma concentration is from 200 to 875 ng/mL. Even further, the maximum total plasma concentration is from 300 to 875 ng/mL. Even further, the maximum total plasma concentration is from 400 to 875 ng/mL. Even further, the maximum total plasma concentration is from 500 to 875 ng/mL. Even further, the maximum total plasma concentration is from 600 to 875 ng/mL. Even further, the maximum total plasma concentration is from 650 to 850 ng/mL. Even further, the maximum total plasma concentration is from 700 to 850 ng/mL. In a further aspect of any of dosage forms as described herein, the dosage form is an oral dosage form. In a still further aspect, the dosage form is an intravenous dosage form. In a further aspect of any of the dosage forms as described herein, the pharmaceutically acceptable salt is a maleate salt. The dosage form is suitable for administration to a mammal, such as a human, particularly for use in the treatment of any of the disorders described herein, such as abnormal cell growth, including cancers, particularly the cancers described herein, and angiogenesis- or VEGF-related ophthalmic disorders.

In a specific embodiment of any of the inventive methods described herein, or for use with any of the inventive dosage forms described herein, particularly in a mammal, such as a human, the abnormal cell growth is cancer, including, but not limited to, lung cancer, bone cancer, pancreatic cancer, skin cancer, cancer of the head or neck, cutaneous or intraocular melanoma, uterine cancer, ovarian cancer, rectal cancer, cancer of the anal region, stomach cancer, colon cancer, breast cancer, uterine cancer, carcinoma of the fallopian tubes, carcinoma of the endometrium, carcinoma of the cervix, carcinoma of the vagina, carcinoma of the vulva, Hodgkin's Disease, cancer of the esophagus, cancer of the small intestine, cancer of the endocrine system, cancer of the thyroid gland, cancer of the parathyroid gland, cancer of the adrenal gland, sarcoma of soft tissue, cancer of the urethra, cancer of the penis, prostate cancer, chronic or acute leukemia, lymphocytic lymphomas, cancer of the bladder, cancer of the kidney or ureter, renal cell carcinoma, carcinoma of the renal pelvis, neoplasms of the central nervous system (CNS), primary CNS lymphoma, spinal axis tumors, brain stem glioma, pituitary adenoma, or a combination of one or more of the foregoing cancers. In another embodiment of said method, said abnormal cell growth is a benign proliferative disease, including, but not limited to, psoriasis, benign prostatic hypertrophy or restinosis.

In a particular aspect of this embodiment, the cancer is selected from gastrointestinal stromal tumors, renal cell carcinoma, breast cancer, colorectal cancer, non-small cell lung cancer, neuroendocrine tumors, small cell lung cancer, mastocytosis, glioma, sarcoma, acute myeloid leukemia, prostate cancer, lymphoma, and combinations thereof.

In further specific embodiments of any of the inventive methods described herein, or for use with any of the inventive dosage forms described herein, the method further comprises administering to the mammal, or the dosage form is further administered with, one or more substances selected from anti-tumor agents, anti-angiogenesis agents, signal transduction inhibitors, and antiproliferative agents, which amounts are together effective in treating said abnormal cell growth. Such substances include those disclosed in PCT publication nos. WO 00/38715, WO 00/38716, WO 00/38717, WO 00/38718, WO 00/38719, WO 00/38730, WO 00/38665, WO 00/37107 and WO 00/38786, the disclosures of which are incorporated herein by reference in their entireties.

Examples of anti-tumor agents include mitotic inhibitors, for example vinca alkaloid derivatives such as vinblastine vinorelbine, vindescine and vincristine; colchines allochochine, halichondrine, N-benzoyltrimethyl-methyl ether colchicinic acid, dolastatin 10, maystansine, rhizoxine, taxanes such as taxol (paclitaxel), docetaxel (Taxotere), 2′-N-[3-(dimethylamino)propyl]glutaramate (taxol derivative), thiocholchicine, trityl cysteine, teniposide, methotrexate, azathioprine, fluorouricil, cytocine arabinoside, 2′2′-difluorodeoxycytidine (gemcitabine), adriamycin and mitamycin. Alkylating agents, for example cis-platin, carboplatin oxiplatin, iproplatin, Ethyl ester of N-acetyl-DL-sarcosyl-L-leucine (Asaley or Asalex), 1,4-cyclohexadiene-1,4-dicarbamic acid, 2,5-bis(1-azirdinyl)-3,6-dioxo-, diethyl ester (diaziquone), 1,4-bis(methanesulfonyloxy)butane (bisulfan or leucosulfan) chlorozotocin, clomesone, cyanomorpholinodoxorubicin, cyclodisone, dianhydroglactitol, fluorodopan, hepsulfam, mitomycin C, hycantheonemitomycin C, mitozolamide, 1-(2-chloroethyl)-4-(3-chloropropyl)-piperazine dihydrochloride, piperazinedione, pipobroman, porfiromycin, spirohydantoin mustard, teroxirone, tetraplatin, thiotepa, triethylenemelamine, uracil nitrogen mustard, bis(3-mesyloxypropyl)amine hydrochloride, mitomycin, nitrosoureas agents such as cyclohexyl-chloroethylnitrosourea, methylcyclohexyl-chloroethylnitrosourea 1-(2-chloroethyl)-3-(2,6-dioxo-3-piperidyl)-1-nitroso-urea, bis(2-chloroethyl)nitrosourea, procarbazine, dacarbazine, nitrogen mustard-related compounds such as mechloroethamine, cyclophosphamide, ifosamide, melphalan, chlorambucil, estramustine sodium phosphate, strptozoin, and temozolamide. DNA anti-metabolites, for example 5-fluorouracil, cytosine arabinoside, hydroxyurea, 2-[(3hydroxy-2-pyrinodinyl)methylene]-hydrazinecarbothioamide, deoxyfluorouridine, 5-hydroxy-2-formylpyridine thiosemicarbazone, alpha-2′-deoxy-6-thioguanosine, aphidicolin glycinate, 5-azadeoxycytidine, beta-thioguanine deoxyriboside, cyclocytidine, guanazole, inosine glycodialdehyde, macbecin II, pyrazolimidazole, cladribine, pentostatin, thioguanine, mercaptopurine, bleomycin, 2-chlorodeoxyadenosine, inhibitors of thymidylate synthase such as raltitrexed and pemetrexed disodium, clofarabine, floxuridine and fludarabine. DNA/RNA antimetabolites, for example, L-alanosine, 5-azacytidine, acivicin, aminopterin and derivatives thereof such as N-[2-chloro-5-[[(2,4-diamino-5-methyl-6-quinazolinyl)methyl]amino]benzoyl]-L-aspartic acid, N-[4-[[(2,4-diamino-5-ethyl-6-quinazolinyl)methyl]amino]benzoyl]-L-aspartic acid, N-[2-chloro-4-[[(2, 4-diaminopteridinyl)methyl]amino]benzoyl]-L-aspartic acid, soluble Baker's antifol, dichloroallyl lawsone, brequinar, ftoraf, dihydro-5-azacytidine, methotrexate, N-(phosphonoacetyl)-L-aspartic acid tetrasodium salt, pyrazofuran, trimetrexate, plicamycin, actinomycin D, cryptophycin, and analogs such as cryptophycin-52 or, for example, one of the preferred anti-metabolites disclosed in European Patent Application No. 239362 such as N-(5-[N-(3,4-dihydro-2-methyl-4-oxoquinazolin-6-ylmethyl)-N-methylamino]-2-thenoyl)-L-glutamic acid; growth factor inhibitors; cell cycle inhibitors; intercalating antibiotics, for example adriamycin and bleomycin; proteins, for example interferon; and anti-hormones, for example anti-estrogens such as Nolvadex™ (tamoxifen) or, for example anti-androgens such as Casodex™ (4′-cyano-3-(4-fluorophenylsulphonyl)-2-hydroxy-2-methyl-3′-(trifluoromethyl)propionanilide). Such conjoint treatment may be achieved by way of the simultaneous, sequential or separate dosing of the individual components of the treatment.

Anti-angiogenesis agents include MMP-2 (matrix-metalloprotienase 2) inhibitors, MMP-9 (matrix-metalloprotienase 9) inhibitors, and COX-II (cyclooxygenase II) inhibitors. Examples of useful COX-II inhibitors include CELEBREX™ (alecoxib), valdecoxib, and rofecoxib. Examples of useful matrix metalloproteinase inhibitors are described in WO 96/33172 (published Oct. 24, 1996), WO 96/27583 (published Mar. 7, 1996), European Patent Application No. 97304971.1 (filed Jul. 8, 1997), European Patent Application No. 99308617.2 (filed Oct. 29, 1999), WO 98/07697 (published Feb. 26, 1998), WO 98/03516 (published Jan. 29, 1998), WO 98/34918 (published Aug. 13, 1998), WO 98/34915 (published Aug. 13, 1998), WO 98/33768 (published Aug. 6, 1998), WO 98/30566 (published Jul. 16, 1998), European Patent Publication 606,046 (published Jul. 13, 1994), European Patent Publication 931,788 (published Jul. 28, 1999), WO 90/05719 (published May 331, 1990), WO 99/52910 (published Oct. 21, 1999), WO 99/52889 (published Oct. 21, 1999), WO 99/29667 (published Jun. 17, 1999), PCT International Application No. PCT/IB98/01113 (filed Jul. 21, 1998), European Patent Application No. 99302232.1 (filed Mar. 25, 1999), Great Britain patent application number 9912961.1 (filed Jun. 3, 1999), U.S. Provisional Application No. 60/148,464 (filed Aug. 12, 1999), U.S. Pat. No. 5,863,949 (issued Jan. 26, 1999), U.S. Pat. No. 5,861,510 (issued Jan. 19, 1999), and European Patent Publication 780,386 (published Jun. 25, 1997), all of which are herein incorporated by reference in their entirety. Preferred MMP-2 and MMP-9 inhibitors are those that have little or no activity inhibiting MMP-1. More preferred, are those that selectively inhibit MMP-2 and/or MMP-9 relative to the other matrix-metalloproteinases (i.e. MMP-1, MMP-3, MMP-4, MMP-5, MMP-6, MMP-7, MMP-8, MMP-10, MMP-11, MMP-12, and MMP-13).

Examples of MMP inhibitors include AG-3340, RO 32-3555, RS 13-0830, and the compounds recited in the following list:

3-[[4-(4-fluoro-phenoxy)-benzenesulfonyl]-(1-hydroxycarbamoyl-cyclopentyl)-amino]-propionic acid; 3-exo-3-[4-(4-fluoro-phenoxy)-benzenesulfonylamino]-8-oxa-bicyclo[3.2.1]octane-3-carboxylic acid hydroxyamide; (2R,3R) 1-[4-(2-chloro-4-fluoro-benzyloxy)-benzenesulfonyl]-3-hydroxy-3-methyl-piperidine-2-carboxylic acid hydroxyamide; 4-[4-(4-fluoro-phenoxy)-benzenesulfonylamino]-tetrahydro-pyran-4-carboxylic acid hydroxyamide; 3-[[4-(4-fluoro-phenoxy)-benzenesulfonyl]-(1-hydroxycarbamoyl-cyclobutyl)-amino]-propionic acid; 4-[4-(4-chloro-phenoxy)-benzenesulfonylamino]-tetrahydro-pyran-4-carboxylic acid hydroxyamide; 3-[4-(4-chloro-phenoxy)-benzenesulfonylamino]-tetrahydro-pyran-3-carboxylic acid hydroxyamide; (2R,3R) 1-[4-(4-fluoro-2-methyl-benzyloxy)-benzenesulfonyl]-3-hydroxy-3-methyl-piperidine-2-carboxylic acid hydroxyamide; 3-[[4-(4-fluoro-phenoxy)-benzenesulfonyl]-(1-hydroxycarbamoyl-1-methyl-ethyl)-amino]-propionic acid; 3-[[4-(4-fluoro-phenoxy)-benzenesulfonyl]-(4-hydroxycarbamoyl-tetrahydro-pyran-4-yl)-amino]-propionic acid; 3-exo-3-[4-(4-chloro-phenoxy)-benzenesulfonylamino]-8-oxa-bicyclo[3.2.1]octane-3-carboxylic acid hydroxyamide; 3-endo-3-[4-(4-fluoro-phenoxy)-benzenesulfonylamino]-8-oxa-bicyclo[3.2.1]octane-3-carboxylic acid hydroxyamide; and 3-[4-(4-fluoro-phenoxy)-benzenesulfonylamino]-tetrahydro-furan-3-carboxylic acid hydroxyamide; and pharmaceutically acceptable salts, solvates and prodrugs of said compounds.

Examples of signal transduction inhibitors include agents that can inhibit EGFR (epidermal growth factor receptor) responses, such as EGFR antibodies, EGF antibodies, and molecules that are EGFR inhibitors; VEGF (vascular endothelial growth factor) inhibitors; and erbB2 receptor inhibitors, such as organic molecules or antibodies that bind to the erbB2 receptor, for example, HERCEPTIN™ (Genentech, Inc. of South San Francisco, Calif., USA).

EGFR inhibitors are described in, for example in WO 95/19970 (published Jul. 27, 1995), WO 98/14451 (published Apr. 9, 1998), WO 98/02434 (published Jan. 22, 1998), and U.S. Pat. No. 5,747,498 (issued May 5, 1998). EGFR-inhibiting agents include, but are not limited to, the monoclonal antibodies C225 and anti-EGFR 22Mab (ImClone Systems Incorporated of New York, N.Y., USA), the compounds ZD-1839 (AstraZeneca), BIBX-1382 (Boehringer Ingelheim), MDX-447 (Medarex Inc. of Annandale, N.J., USA), and OLX-103 (Merck & Co. of Whitehouse Station, N.J., USA), VRCTC-310 (Ventech Research) and EGF fusion toxin (Seragen Inc. of Hopkinton, Mass.).

VEGF inhibitors, for example AG-13736 (Pfizer, Inc.), can also be combined or co-administered with the composition. VEGF inhibitors are described in, for example in WO 99/24440 (published May 20, 1999), PCT International Application PCT/IB99/00797 (filed May 3, 1999), in WO 95/21613 (published Aug. 17, 1995), WO 99/61422 (published Dec. 2, 1999), U.S. Pat. No. 5,834,504 (issued Nov. 10, 1998), WO 98/50356 (published Nov. 12, 1998), U.S. Pat. No. 5,883,113 (issued Mar. 16, 1999), U.S. Pat. No. 5,886,020 (issued Mar. 23, 1999), U.S. Pat. No. 5,792,783 (issued Aug. 11, 1998), U.S. Pat. No. 6,534,524, WO 99/10349 (published Mar. 4, 1999), WO 97/32856 (published Sep. 12, 1997), WO 97/22596 (published Jun. 26, 1997), WO 98/54093 (published Dec. 3, 1998), WO 98/02438 (published Jan. 22, 1998), WO 99/16755 (published Apr. 8, 1999), and WO 98/02437 (published Jan. 22, 1998), all of which are herein incorporated by reference in their entirety. Other examples of some specific VEGF inhibitors are IM862 (Cytran Inc. of Kirkland, Wash., USA); Avastin™ or bevacizumab, an anti-VEGF monoclonal antibody (Genentech, Inc. of South San Francisco, Calif.); and angiozyme, a synthetic ribozyme from Ribozyme (Boulder, Colo.) and Chiron (Emeryville, Calif.).

ErbB2 receptor inhibitors, such as GW-282974 (Glaxo Wellcome plc), and the monoclonal antibodies AR-209 (Aronex Pharmaceuticals Inc. of The Woodlands, Tex., USA) and 2B-1 (Chiron), may be administered in combination with the composition. Such erbB2 inhibitors include those described in WO 98/02434 (published Jan. 22, 1998), WO 99/35146 (published Jul. 15, 1999), WO 99/35132 (published Jul. 15, 1999), WO 98/02437 (published Jan. 22, 1998), WO 97/13760 (published Apr. 17, 1997), WO 95/19970 (published Jul. 27, 1995), U.S. Pat. No. 5,587,458 (issued Dec. 24, 1996), and U.S. Pat. No. 5,877,305 (issued Mar. 2, 1999), each of which is herein incorporated by reference in its entirety. ErbB2 receptor inhibitors useful in the present invention are also described in U.S. Provisional Application No. 60/117,341, filed Jan. 27, 1999, and in U.S. Provisional Application No. 60/117,346, filed Jan. 27, 1999, both of which are herein incorporated by reference in their entirety.

Other antiproliferative agents that may be used include inhibitors of the enzyme farnesyl protein transferase and inhibitors of the receptor tyrosine kinase PDGFr, including the compounds disclosed and claimed in the following U.S. patent application Ser. Nos. 09/221,946 (filed Dec. 28, 1998); 09/454,058 (filed Dec. 2, 1999); 09/501,163 (filed Feb. 9, 2000); 09/539,930 (filed Mar. 31, 2000); 09/202,796 (filed May 22, 1997); 09/384,339 (filed Aug. 26, 1999); and 09/383,755 (filed Aug. 26, 1999); and the compounds disclosed and claimed in the following United States provisional patent applications: 60/168,207 (filed Nov. 30, 1999); 60/170,119 (filed Dec. 10, 1999); 60/177,718 (filed Jan. 21, 2000); 60/168,217 (filed Nov. 30, 1999), and 60/200,834 (filed May 1, 2000). Each of the foregoing patent applications and provisional patent applications is herein incorporated by reference in their entirety.

The compound of formula 1, or pharmaceutically acceptable salts or solvates thereof, may also be used with other agents useful in treating abnormal cell growth or cancer, including, but not limited to, agents capable of enhancing antitumor immune responses, such as CTLA4 (cytotoxic lymphocite antigen 4) antibodies, and other agents capable of blocking CTLA4; and anti-proliferative agents such as other farnesyl protein transferase inhibitors. Specific CTLA4 antibodies that can be used in the present invention include those described in U.S. Provisional Application 60/113,647 (filed Dec. 23, 1998) which is herein incorporated by reference in its entirety.

Specific examples of combination therapy can be found in PCT Publication No. WO 03/015608 and WO 04/045523 (U.S. Patent Publication No. 2004-0152759), the disclosures of which are incorporated herein by reference in their entireties.

The invention also includes methods of using isotopically-labeled compounds, which are identical to those recited in compound of formula 1, but for the fact that one or more atoms are replaced by an atom having an atomic mass or mass number different from the atomic mass or mass number usually found in nature. Examples of isotopes that can be incorporated into a compound of formula 1 include isotopes of hydrogen, carbon, nitrogen, oxygen, phosphorus, sulfur, fluorine and chlorine, such as ²H, ³H, ³C, ¹⁴C, ¹⁵N, ¹⁸O, ¹⁷O, ³¹P, ³²P, ³⁵S, ¹⁸F, and ³⁶Cl, respectively. Methods of using a compound of formula 1, or a pharmaceutically acceptable salt or solvate thereof, which contain the aforementioned isotopes and/or other isotopes of other atoms are within the scope of this invention. Certain isotopically-labeled compounds, for example those into which radioactive isotopes such as ³H and ¹⁴C are incorporated, are useful in drug and/or substrate tissue distribution assays. Tritiated, i.e., ³H, and carbon-14, i.e., ¹⁴C, isotopes are particularly preferred for their ease of preparation and detectability. Further, substitution with heavier isotopes such as deuterium, i.e., ²H, can afford certain therapeutic advantages resulting from greater metabolic stability, for example increased in vivo half-life or reduced dosage requirements and, hence, may be preferred in some circumstances. Isotopically labeled compound of formula 1, or a pharmaceutically acceptable salt or solvate thereof, can generally be prepared by carrying out the procedures described for the non-labeled compound, substituting a readily available isotopically labeled reagent for a non-isotopically labeled reagent.

### DEFINITIONS

“Abnormal cell growth”, as used herein, unless otherwise indicated, refers to cell growth that is independent of normal regulatory mechanisms (e.g., loss of contact inhibition). This includes the abnormal growth of: (1) tumor cells (tumors) that proliferate by expressing a mutated tyrosine kinase or overexpression of a receptor tyrosine kinase; (2) benign and malignant cells of other proliferative diseases in which aberrant tyrosine kinase activation occurs; and (4) any tumors that proliferate by receptor tyrosine kinases.

The term “treating”, as used herein, unless otherwise indicated, means reversing, alleviating, inhibiting the progress of, or preventing the disorder or condition to which such term applies, or one or more symptoms of such disorder or condition. The term “treatment”, as used herein, unless otherwise indicated, refers to the act of treating as “treating” is defined immediately above.

The phrase “pharmaceutically acceptable salt(s)”, as used herein, unless otherwise indicated, includes salts of acidic or basic groups which may be present in a compound. Compounds that are basic in nature are capable of forming a wide variety of salts with various inorganic and organic acids. The acids that may be used to prepare pharmaceutically acceptable acid addition salts of such basic compounds are those that form non-toxic acid addition salts, i.e., salts containing pharmacologically acceptable anions, such as the acetate, benzenesulfonate, benzoate, bicarbonate, bisulfate, bistosylate, bitartrate, borate, bromide, calcium edetate, camsylate, carbonate, chloride, clavulanate, citrate, dihydrochloride, edetate, edislyate, estolate, esylate, ethylsuccinate, fumarate, gluceptate, gluconate, glutamate, glycollylarsanilate, hexylresorcinate, hydrabamine, hydrobromide, hydrochloride, iodide, isothionate, lactate, lactobionate, laurate, malate, maleate, mandelate, mesylate, methylsulfate, mucate, napsylate, nitrate, oleate, oxalate, pamoate (embonate), palmitate, pantothenate, phospate/diphosphate, polygalacturonate, salicylate, stearate, subacetate, succinate, tannate, tartrate, teoclate, tosylate, triethiodode, and valerate salts. Particularly preferred salts include maleate salts.

The term “prodrug”, as used herein, unless otherwise indicated, means compounds that are drug precursors, which following administration, release the drug in vivo via some chemical or physiological process (e.g., a prodrug on being brought to the physiological pH is converted to the desired drug form).

“Continuous dosing schedule”, as used herein, unless otherwise indicated, refers to a dosing schedule wherein compound of formula 1, or a dosage form comprising the compound of formula 1, is administered during a treatment period without a rest period. Throughout the treatment period of a continuous dosing schedule, the compound of formula 1, or a dosage form comprising the compound of formula 1, can be administered, for example, daily, or every other day, or every third day. On a day when compound of formula 1, or a dosage form comprising the compound of formula 1 is administered, it can be administered in a single dose, or in multiple doses throughout the day.

“Intermittent dosing schedule”, as used herein, unless otherwise indicated, refers to a dosing schedule that comprises a treatment period and a rest period. Throughout the treatment period of an intermittent dosing schedule, compound of formula 1, or a dosage form comprising the compound of formula 1, can be administered, for example, daily, or every other day, or every third day. On a day when compound of formula 1, or a dosage form comprising the compound of formula 1, is administered, it can be administered in a single dose, or in multiple doses throughout the day. During the rest period, compound of formula 1, or a dosage form comprising the compound of formula 1 is not administered. In an intermittent dosing regimen, the treatment period is typically from 10 to 30 days, such as 2, 3 or 4 weeks, and the rest period is typically from 3 to 15 days, such as 1 or 2 weeks. The combination of any treatment period from 10 to 30 days with any rest period from 3 to 15 days is contemplated. Intermittent dosing regimens can be expressed as treatment period in weeks/rest period in weeks. For example, a 4/1 intermittent dosing schedule refers to an intermittent dosing schedule wherein the treatment period is four weeks and the rest period is one week. A 4/2 intermittent dosing schedule refers to an intermittent dosing schedule wherein the treatment period is four weeks and the rest period is two weeks. Similarly, a 3/1 intermittent dosing schedule refers to an intermittent dosing schedule wherein the treatment period is three weeks and the rest period is one week.

Complete Response (CR), as used herein, unless otherwise indicated, refers to disappearance of all measurable and nonmeasurable lesions and no appearance of new lesions in a patient under the treatment of compound of formula 1, its pharmaceutically acceptable salt or solvate thereof, or a mixture thereof.

Partial Response (PR), as used herein, unless other wise indicated, refers to at least a 30% decrease in the sum of the LDs of target lesions (taking as reference the baseline sum), without progression of nontarget lesions and no appearance of new lesions in a patient under treatment of compound of formula 1, its pharmaceutically acceptable salt or solvate thereof, or a mixture thereof.

It should be further appreciated that dosing regimens can be adjusted by one skilled in the art to more conveniently accommodate coordination of the dosing regimens of a compound of formula 1, or a pharmaceutically acceptable salt or solvate thereof, and additional therapeutic agents, if such adjustments are therapeutically acceptable. For example, if an additional therapeutic agent were administered as an infusion once every 4 weeks, a dosing regimen of a compound of formula 1, or a pharmaceutically acceptable salt or solvate thereof, of 3/1 or 2/2, or a continuous dosing regimen, would best coordinate with the regimen of the additional therapeutic agent.

As used herein, “a compound of formula 1” or “compound 1” refers to 5-[(Z)-(5-fluoro-2-oxo-1,2-dihydro-3H-indol-3-ylidene)methyl]-N-[(2S)-2-hydroxy-3-morpholin-4-ylpropyl]-2,4-dimethyl-1H-pyrrole-3-carboxamide. It should also be understood that any reference to “a compound of formula 1” or “compound 1” or “5-[(Z)-(5-fluoro-2-oxo-1,2-dihydro-3H-indol-3-ylidene)methyl]-N-[(2S)-2-hydroxy-3-morpholin-4-ylpropyl]-2,4-dimethyl-1H-pyrrole-3-carboxamide” also refers to any pharmaceutically acceptable salt or solvate thereof, or to mixtures thereof. Preferably, the pharmaceutically acceptable salt is a maleate salt.

References to amounts of a compound of formula 1 refer to free base equivalent amounts. For example, if a compound of formula 1 is used in the form of a salt, reference to “50 mg of compound 1” or “50 mg of compound 1, free base equivalent” means the amount of salt that would be needed to provide 50 mg of the free base upon complete dissociation of the salt.

As used herein, “Cₘₐₓ” refers to the maximum plasma concentration; tₘₐₓ refers to the time when the Cₘₐₓ occurs following administering the dosage; AUC refers to area under the plasma concentration-time curve from time zero to infinity; t_{1/2} refers to plasma elimination half-life; % CV refers to percent coefficient of variation; C_{(trough 24 h)} refers to trough plasma concentration at 24 hours after dosing; and QD indicates once daily.

### DETAILED DESCRIPTION OF THE INVENTION

The compound of formula 1, or pharmaceutically acceptable salts and solvates thereof, can be prepared as described in U.S. Pat. No. 6,653,308, WO03/070723 (US 2003/0092917) and WO2005-033098 (US 2005-0118255), which are incorporated herein by reference. Certain starting materials may be prepared according to methods familiar to those skilled in the art and certain synthetic modifications may be done according to methods familiar to those skilled in the art. Preferred formulations of compound 1 are disclosed in WO 04/024127 (US 2004/229930), which is incorporated herein by reference.

The compound of formula 1 is capable of forming a wide variety of different salts with various inorganic and organic acids. Although such salts must be pharmaceutically acceptable for administration to mammals, it is often desirable in practice to initially isolate the compound of formula 1 from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid. In particular, the compound of formula 1 forms a maleate salt, as described in WO2005-033098 (US 2005-0118255), which is convenient for administration to mammals.

Administration of the compound of formula 1, or a pharmaceutically acceptable salt or solvate thereof, can be effected by any method that enables delivery of the compound to the site of action. These methods include oral routes, intraduodenal routes, parenteral injection (including intravenous, subcutaneous, intramuscular, intravascular or infusion, intra-occular (topical, conjuctival, intra-vitreal, or sub-Tenon), topical, and rectal administration.

The compound may, for example, be provided in a form suitable for oral administration as a tablet, capsule, pill, powder, sustained release formulation, solution, suspension, for parenteral injection as a sterile solution, suspension or emulsion, for topical administration as an ointment or cream or for rectal administration as a suppository. The compound may be in unit dosage forms suitable for single administration of precise dosages. Preferably, dosage forms include a conventional pharmaceutical carrier or excipient and the compound of formula 1, or a pharmaceutically acceptable salt or solvate thereof, as an active ingredient. In addition, dosage forms may include other medicinal or pharmaceutical agents, carriers, adjuvants, etc. Preferred formulations of a compound of formula 1 are disclosed in WO 04/024127 (US 2004/229930).

Exemplary parenteral administration forms include solutions or suspensions in sterile aqueous solutions, for example, aqueous propylene glycol or dextrose solutions. Such dosage forms can be suitably buffered, if desired.

Suitable pharmaceutical carriers include inert diluents or fillers, water and various organic solvents. The pharmaceutical composition may, if desired, contain additional ingredients such as flavorings, binders, excipients and the like. Thus for oral administration, tablets containing various excipients, such as citric acid may be employed together with various disintegrants such as starch, alginic acid and certain complex silicates and with binding agents such as sucrose, gelatin and acacia. Additionally, lubricating agents such as magnesium stearate, sodium lauryl sulfate and talc are often useful for tableting purposes. Solid compositions of a similar type may also be employed in soft and hard filled gelatin capsules. Preferred materials therefor include lactose or milk sugar and high molecular weight polyethylene glycols. When aqueous suspensions or elixirs are desired for oral administration the active compound therein may be combined with various sweetening or flavoring agents, coloring matters or dyes and, if desired, emulsifying agents or suspending agents, together with diluents such as water, ethanol, propylene glycol, glycerin, or combinations thereof.

In preferred embodiments of the dosage forms of the invention, the dosage form is an oral dosage form, more preferably, a tablet or a capsule.

In preferred embodiments of the methods of the invention, the compound of formula 1, or a pharmaceutically acceptable salt or solvate thereof, is administered orally, such as, for example, using an oral dosage form as described in U.S. Patent Publication No. US 2004/229,930 and corresponding PCT Publication No. WO 04/024127.

The methods include administering the compound of formula 1, or a pharmaceutically acceptable salt or solvate thereof, using any desired dosage regimen. In one specific embodiment, the compound is administered once per day (quaque die, or QD), or twice per day (bis in die, or BID), although more or less frequent administration is within the scope of the invention. The compound can be administered to the mammal, including a human, in a fed or fasted state, preferably in a fasted state (no food or beverage within 2 hours before and after administration).

Methods of preparing various dosage forms with a specific amount of the compound of formula 1 are known, or will be apparent, to those skilled in this art. For examples, see Remington's Pharmaceutical Sciences, Mack Publishing Company, Easter, Pa., 15th Edition (1975).

Cₘₐₓ values, or maximum total plasma concentrations, of a compound of formula 1 can be measured according to techniques well known to those skilled in the art. For example, after a compound of formula 1 has been administered to a mammal, blood samples can be taken at fixed time points over a period of time (e.g. 24 hours) and the serum or plasma concentration of compound of formula 1 can be measured using standard analytical techniques known in the art. In vivo determinations Cₘₐₓ can then be made by plotting the serum or plasma concentration of compound of formula 1 along the ordinate (y-axis) against time along the abscissa (x-axis).

### EXAMPLES

Particular aspects of the present invention can be further described by reference to the examples below. The examples below are intended to illustrate particular embodiments of the present invention and are not meant to limit the scope of the invention in any way.

### Example 1

### In Vivo Study in Patients with Solid Tumor

The maleate salt of compound 1 was administered to human patients with solid tumor malignancies not amenable to conventional therapies in a Phase I dose-escalating multicenter study. The types of tumor malignancies included colorectal carcinoma, renal cell carcinoma, esophageal carcinoma, thymus carcinoma, mastocytosis, lung cancer and multiple endocrine neoplasia type II. Patients were treated in cohorts of 6 with escalating QD (once per day) doses of the maleate salt of compound 1 under fasting conditions. Each study cycle of 5 weeks consisted of 4 weeks of treatment followed by 1 week of rest (4/1 schedule), or continuous dosing without any rest period.

Full pharmacokinetic profiles were collected on Cycle 1 Day 1 (C1D1), Cycle 1 Day 28 (C1D28), and Cycle 2 Day 28 (C2D28). Preliminary pharmacokinetic parameters for the first 44 patients, i.e. the first 7 dosing groups, were estimated using nominal collection times and quality-controlled, non-quality assured bioanalytical data. These data are summarized in Table 1A and Table 1B. The dosage amounts in Table 1A and Table 1B are free base equivalent amounts.

TABLE 1A
Preliminary Mean (% CV) Plasma Pharmacokinetic
Parameters in Subjects with Solid Tumors

| Dosing | Cycle and | Cₘₐₓ | tₘₐₓ | AUC₍₀₋₂₄₎ | t_{½} | C_{(trough, 24 h)} |
|---|---|---|---|---|---|---|
| schedule | Study Day | (ng/mL) | (h) | (ng · h/mL) | (h) | (ng/mL) |
| 25 mg QD | C1D1 (n = 6) | 55.1 (65) | 2.8 (125) | 547 (29) | 11.9 (22) | 13.8 (41) |
| (4/1) | C1D28 (n = 6) | 72.9 (34) | 2.7 (57) | 809 (37) | 24.8 (32) | 21.1 (52) |
| | C2D28 (n = 5) | 61.8 (53) | 3.2 (34) | 794 (47) | 17.7 (30) | 21.4 (54) |
| 50 mg QD | C1D1 (n = 6) | 84.6 (47) | 2.8 (47) | 888 (47) | 10.8 (32) | 20.8 (84) |
| (4/1) | C1D28 (n = 5) | 126.2 (48) | 6.6 (148) | 1348 (52) | 24.0 (29) | 50.8 (123) |
| | C2D28 (n = 3) | 173.0 (81) | 3.3 (125) | 1794 (80) | 18.9 (33) | 44.2 (95) |
| 100 mg QD | C1D1 (n = 7) | 270.3 (53) | 2.6 (44) | 2479 (46) | 12.5 (79) | 86.8 (114) |
| (4/1) | C1D28 (n = 6) | 413.4 (82) | 2.7 (31) | 6054 (107) | 22.0 (46) | 110.9 (133) |
| | C2D28 (n = 4) | 180.9 (82) | 13.5 (90) | 2802 (77) | 23.0 (56) | 156.4 (109) |
| 150 mg QD | C1D1 (n = 7) | 421.1 (51) | 1.6 (34) | 3373 (54) | 13.6 (59) | 81.7 (101) |
| (4/1) | C1D28 (n = 5) | 299.2 (37) | 1.8 (72.4) | 2958 (59) | 19.3 (35) | 75.8 (83) |
| | C2D28 (n = 4) | 268.9 (45) | 3.0 (39) | 3094 (51) | 13.2 (42) | 54.6 (64) |
| 200 mg QD | C1D1 (n = 6) | 262.5 (88) | 8.7 (96.2) | 3435 (79) | 18.8 (58) | 147.0 (125) |
| (4/1) | C1D28 (n = 5) | 560.6 (40) | 6.6 (121) | 8207 (33) | 26.9 (32) | 259.0 (45) |
| | C2D28 (n = 5) | 375.2 (36) | 5.6 (77) | 6269 (43) | 15.3 (8) | 211.2 (56) |
| 250 mg QD | C1D1 (n = 6) | 848.3 (36) | 3.7 (66) | 7581 (33) | 11.5 (57) | 142.9 (76) |
| (4/1) | C1D28 (n = 4) | 733.5 (39) | 4.5 (43) | 9812 (26) | 22.4 (6) | 245.5 (51) |
| | C2D28 (n = 4) | 748.5 (71) | 5.5 (55) | 9770 (54) | 13.2 (25) | 278.8 (84) |
| 100 mg QD | C1D1 (n = 6) | 170.9 (42) | 4.3 (32) | 1802 (44) | 12.9 (32) | 34.1 (67) |
| (continuous) | C1D28 (n = 6) | 254.7 (21) | 3.3 (16) | 2988 (28) | 11.4 (43) | 63.0 (36) |
| | C2D28 (n = 4) | 250.0 (33) | 3.7 (73) | 3234 (22) | 13.8 (45) | 62.8 (42) |

TABLE 1B
Mean plasma concentration (ng/mL) following the last dose on cycle 1 day 28

| Nominal | 25 mg QD | 50 mg QD | 100 mg QD | 150 mg QD | 200 mg QD | 250 mg QD | 100 mg QD | 150 mg QD |
|---|---|---|---|---|---|---|---|---|
| Time | 4/1 | 4/1 | 4/1 | 4/1 | 4/1 | 4/1 | Continuous | Continuous |
| (h) | (n = 6) | (n = 5) | (n = 6) | (n = 5) | (n = 5) | (n = 6) | (n = 4) | (n = 6) |
| 0 | 22.23 | 37.22 | 120.71 | 99.82 | 352.60 | 64.54 | 283.68 | 123.53 |
| 1 | 50.13 | 72.27 | 218.27 | 184.36 | 407.80 | 106.60 | 231.50 | 243.87 |
| 2 | 46.72 | 84.49 | 325.83 | 251.80 | 408.20 | 176.88 | 524.00 | 452.73 |
| 3 | 46.95 | 85.64 | 336.83 | 267.96 | 395.60 | 250.00 | 459.50 | 407.13 |
| 4 | 58.28 | 81.10 | 344.47 | 230.80 | 441.40 | 200.50 | 560.00 | 519.17 |
| 6 | 50.27 | 71.30 | 312.50 | 160.76 | 463.40 | 157.00 | 707.00 | 471.00 |
| 8 | 41.28 | 68.60 | 261.37 | 142.08 | 348.60 | 161.17 | 395.33 | 404.50 |
| 10 | 36.75 | 59.38 | 207.70 | 131.64 | 361.20 | 141.08 | 565.50 | 345.00 |
| 12 | 30.10 | 50.43 | 203.50 | 91.26 | 355.25 | 130.27 | 377.00 | 465.33 |
| 20 | 19.49 | 30.50 | 311.45 | 42.91 | 189.00 | 53.63 | 264.67 | 104.47 |
| 24 | 21.08 | 24.28 | 110.94 | 94.75 | 259.00 | 62.97 | 245.50 | 270.60 |
| 48 | 6.24 | 25.36 | 60.79 | 31.20 | 87.64 | | | |
| 72 | 2.61 | 7.89 | 16.08 | 9.08 | 31.40 | | | |
| 96 | 1.58 | 16.96 | 9.96 | 4.17 | 12.96 | | | |
| 144 | 0.63 | 3.27 | 2.94 | 1.52 | 4.77 | | | |

In calculating the C2D28 data for the 25 mg QD 4/1 dosing schedule, the data of one patient was excluded who had unusually high plasma concentrations (Cₘₐₓ=394 ng/mL; AUC₍₀₋₂₄₎=6997 ng·h/mL); the reason for the approximately 5-fold higher exposures on C2D28 compared with C1D28 in this patient is unknown.

Compound of formula 1 administered in the fasted state was absorbed within the first 6 hours after dosing. The mean terminal plasma half-life (t_{1/2}) over 24 hours after dosing on C1D1 ranged from 10.8 to 18.8 hours. For patients in the 4/1 dosing schedule, upon collection of blood samples for 144 hours (through washout period) after the last dose on Day 28 of the dosing cycle, a longer t_{1/2} was identified; mean estimates for this t_{1/2} ranged from 13.2 to 26.9 hours across the dose groups. This longer elimination phase occurred late, usually about 72 hours after dosing, and after plasma concentrations had already significantly declined. There was no change in the overall plasma elimination profile for the drug across the 25- to 250-mg groups evaluated to date.

Based on the effective t_{1/2}, there was no unexpected accumulation of the drug with continuous dosing in most subjects, as seen from the plasma exposures on Day 28 of dosing. Also, when comparing the Cₘₐₓ of C1D28 of 100 mg QD continuous, with Cₘₐₓ of C2D28 of 100 mg QD continuous in Table 1A, the data showed that there was no drug accumulation in the plasma of the patients undergoing 100 mg QD continuous dosing from cycle 1 to cycle 2. The same conclusion was drawn when AUC₀₋₂₄ of C1D28 and that of C2D28 of the 100 mg QD continuous in Table 1A were compared.

Steady state was anticipated within the first week of dosing. As shown in Table 1B, extrapolating beyond the measured plasma concentrations at 144 hours (during the rest period after the last dose of Cycle 1) indicated that compound of formula 1 concentrations declined to negligible levels (<5 ng/mL) prior to the start of dosing in the following cycle.

Data reported here from the first 44 patients demonstrated generally dose-linear pharmacokinetics. For example, according to data in Table 1A, the steady state C2D28 mean AUC₍₀₋₂₄₎ was 794 and 9770 ng h/mL for the 25- and 250-mg dosing cohorts (4/1 schedule), respectively, which represented AUC₍₀₋₂₄₎ increments of 1:12 for dose increments of 1:10 respectively. Also for example, according to Table 1B, the mean plasma concentration at a certain time point, of compound of formula 1 was roughly proportionally to the amount of compound 1 administered. For example, at hour 4, the mean plasma concentration of 25 mg QD 4/1, 50 mg QD 4/1 and 150 mg QD 4/1 is 58.28, 81.10 and 230.8 ng/mL respectively.

In summary, compound of formula 1 plasma pharmacokinetics in this study in patients with solid tumors indicated absorption of the drug in the first 6 hours after dosing, followed by elimination from plasma with an effective t_{1/2} of 11 to 19 hours. There was no unexpected drug accumulation with continuous dosing compared to dosing on the 4/1 schedule.

### Example 2

### Efficacy Study in Humans with Solid Tumors

50 patients were treated under a dose-escalating multicenter study of patients with solid tumor malignancies not amenable to conventional therapies. The types of tumor malignancies that the patients had included colorectal carcinoma, renal cell carcinoma, esophageal carcinoma, thymus carcinoma, mastocytosis, lung cancer and multiple endocrine neoplasia type II and other malignances. Patients were treated in cohorts of 6 with escalating QD (once per day) doses of a maleate salt of a compound of formula 1. Each study cycle was a five week cycle consisting of 4 weeks of treatment followed by 1 week of rest (4/1 schedule) or a five week cycle of continuous dosing without any rest period.

Of these 50 patients, all patients were evaluated for efficacy determinations. Tumor size was measured at the end of each cycle of treatment. Among the 50 patients, 1 patient showed complete response and 7 patients showed partial response of tumor shrinkage of up to 30% in volume. The partial response of four of these seven patients was confirmed by a repeat assessment four weeks later. The partial response of the other three patients has not been confirmed. The tumor shrinkage was determined by either CT scan or MRI as per RECIST criteria. These results are summarized in table 2.

TABLE 2
Efficacy study in humans with solid tumors

| Patient | | | Cycle 2 and | Response time |
|---|---|---|---|---|
| number | Tumor Type | Cycle 1 | beyond | and response |
| 1 | Renal cell carcinoma | 50 mg 4/1 QD | 50 mg 4/1 QD | After 2nd cycle, PR; CR |
| | | | | at cycle 5 |
| 2 | Biliary cell Carcinoma | 100 mg 4/1 QD | 100 mg 4/1 QD | PR at cycle 5, confirmed. |
| 3 | Thyroid carcinoma | 150 mg QD 4/1 | 150 mg QD 4/1 | PR after 1^{st} cycle, |
| | | | | confirmed. |
| 4 | Colon | 150 mg QD 4/1 | 150 mg QD 4/1 | PR after 1^{st} cycle, not |
| | adenocarcinoma | | | confirmed. |
| 5 | Renal cell carcinoma | 250 mg QD 4/1 | 250 mg QD 4/1 | PR after 1^{st} cycle, |
| | | | | confirmed. |
| 6 | Alveolar soft tissue | 250 mg QD 4/1 | 200 mg QD 4/1 | PR, not confirmed |
| | carcinoma | | | |
| 7 | Renal cell carcinoma | 150 mg | 150 mg | PR after 1^{st} cycle, not |
| | | continuous | continuous | confirmed |
| 8 | Thymoma | 150 mg | 100 mg | PR, not confirmed. |
| | | continuous | continuous | |

In Table 2, PR means partial response, CR means complete response. During the 2^{nd} cycle of treatment of patient number 1, patient mistakenly increased the amount taken to 100 mg free base equivalent for a few days.

All references cited herein, including patents, patent applications, publications and priority documents, are incorporated herein by reference in their entireties.

1. A method of treating cancer in a patient, comprising administering to the patient a compound of formula 1: 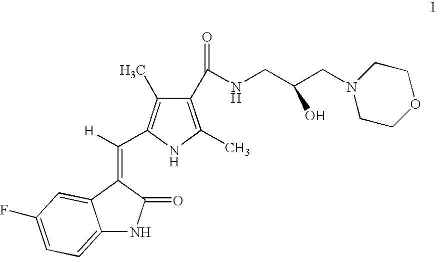
or a pharmaceutically acceptable salt or solvate thereof, or a mixture thereof, in an amount of from 5 to 300 mg free base equivalent per day.

2. The method of claim 1, wherein the cancer is selected from the group consisting of a gastrointestinal stromal tumor, renal cell carcinoma, biliary cell carcinoma, thyroid carcinoma, colon adenocarcinoma, alveolar soft tissue carcinoma, thymoma, breast cancer, colorectal cancer, non-small cell lung cancer, a neuroendocrine tumor, small cell lung cancer, mastocytosis, glioma, sarcoma, acute myeloid leukemia, prostate cancer, lymphoma, and pancreatic cancer.

3. The method of claim 1, wherein the amount is from 50 to 250 mg free base equivalent.

4. The method of claim 1, wherein the amount is from 100 to 200 mg free base equivalent.

5. The method of claim 1, wherein the amount is 150 mg free base equivalent or 200 mg free base equivalent.

6. The method of claim 1, wherein the amount is administered on a continuous dosing schedule.

7. The method of any of claim 1, wherein the amount is administered on an intermittent dosing schedule.

8. The method of claim 7, wherein the intermittent dosing schedule comprises a treatment period of from 2 to 4 weeks and a rest period of from 1 to 2 weeks.

9. A method of treating an angiogenesis- or VEGF-related ophthalmic disorder in a patient, comprising administering to the patient a compound of formula 1, or a pharmaceutically acceptable salt or solvate thereof, or a mixture thereof, in an amount of from 5 to 300 mg free base equivalent per day.

10. The method of claim 9, wherein the ophthalmic disorder is age related macular degeneration, choroidal neovascularization, retinopathy, retinitis, uveitis, retinal vein occlusion, iris neovascularization, corneal neovascularization, macular edema, or neovascular glaucoma.

11. A dosage form comprising a compound of formula 1: 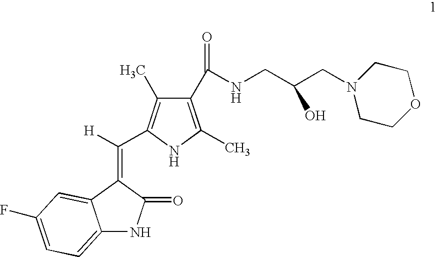
or a pharmaceutically acceptable salt or solvate thereof, or a mixture thereof, in an amount of from 5 to 300 mg free base equivalent.

12. The dosage form of claim 11, wherein the amount is from 25 to 300 mg free base equivalent.

13. The dosage form of claim 11, wherein the amount is from 50 to 250 mg free base equivalent.

14. The dosage form of claim 11, wherein the amount is from 100 to 200 mg free base equivalent.

15. The dosage form of claim 11, wherein the dosage form is an oral dosage form.
